(19) United States
(12) Patent Application Publication
(10) Pub. No.: US 2016/0278741 A1
(43) Pub. Date: Sep. 29, 2016

(54) APPARATUS AND METHOD OF MEASURING ELASTICITY USING ULTRASOUND

(71) Applicant: SAMSUNG MEDISON CO., LTD., Hongcheon-gun (KR)

(72) Inventors: Sun-mo YANG, Hwaseong-si (KR); Yong-soo KIM, Seoul (KR); Seung-ju LEE, Ansan-si (KR)

(73) Assignee: SAMSUNG MEDISON CO., LTD., Hongcheon-gun (KR)

(21) Appl. No.: 14/987,836

(22) Filed: Jan. 5, 2016

Related U.S. Application Data
(60) Provisional application No. 62/137,424, filed on Mar. 24, 2015.

(30) Foreign Application Priority Data
Jun. 2, 2015 (KR) 10-2015-0078240

(51) Int. Cl. A61B 8/08; A61B 8/00

(52) U.S. Cl. CPC A61B 8/485; A61B 8/461; A61B 8/54; A61B 8/5223

(57) ABSTRACT

Provided is a method of operating an ultrasound diagnosis apparatus, including: acquiring a plurality of pieces of elasticity data for an object; acquiring a plurality of measurement values respectively from the plurality of pieces of elasticity data; displaying the plurality of measurement values; selecting at least one of the plurality of measurement values; displaying the selected at least one measurement value to be distinguished from non-selected measurement values among the plurality of measurement values; and displaying a resultant value calculated based on the selected at least one measurement value.

### CROSS-REFERENCE TO RELATED APPLICATIONS

This application claims the benefit of U.S. Provisional Application No. 62/137,424, filed on Mar. 24, 2015, in the US Patent Office and Korean Patent Application No. 10-2015-0078240, filed on Jun. 2, 2015, in the Korean Intellectual Property Office, the disclosures of which are incorporated herein in their entireties by reference.

### BACKGROUND

1. Field

The present disclosure relates to methods and apparatuses for measuring elasticity using ultrasound, and more particularly, to methods and apparatuses for measuring elasticity of an object by using an ultrasound diagnosis device.

2. Description of the Related Art

Ultrasound diagnosis apparatuses transmit ultrasound signals generated by transducers of a probe to an object and receive echo signals reflected from the object, thereby obtaining at least one image of an internal part of the object (e.g., soft tissues or blood flow). In particular, ultrasound diagnosis apparatuses are used for medical purposes including observation of the interior of an object, detection of foreign substances, and diagnosis of damage to the object. Such ultrasound diagnosis apparatuses provide high stability, display images in real time, and are safe due to the lack of radioactive exposure, compared to X-ray apparatuses. Therefore, ultrasound diagnosis apparatuses are widely used together with other image diagnosis apparatuses including a computed tomography (CT) apparatus, a magnetic resonance imaging (MRI) apparatus, and the like

### SUMMARY

Provided are methods and apparatuses for more easily providing a user with an elasticity measurement of an object.

Additional aspects will be set forth in part in the description which follows and, in part, will be apparent from the description, or may be learned by practice of the presented exemplary embodiments.

According to an aspect of an exemplary embodiment, a method of operating an ultrasound diagnosis apparatus includes: acquiring a plurality of pieces of elasticity data for an object; acquiring a plurality of measurement values respectively from the plurality of pieces of elasticity data; displaying the plurality of measurement values; selecting at least one of the plurality of measurement values; displaying the selected at least one measurement value to be distinguished from non-selected measurement values among the plurality of measurement values; and displaying a resultant value calculated based on the selected at least one measurement value.

The plurality of measurement values may include a plurality of elasticity values respectively acquired from the plurality of pieces of elasticity data. The displaying of the plurality of measurement values may include determining at least one reference value determined based on the plurality of elasticity values respectively acquired from the plurality of pieces of elasticity data and displaying a graph representing the determined at least one reference value and the plurality of measurement values.

The at least one reference value may include at least one of a median value, a first quartile, and a third quartile for the plurality of elasticity values.

The displayed graph may include at least one baseline representing a magnitude of the at least one reference value.

The resultant value may include at least one of a median value, an average value, a reliability index, a standard deviation, and a ratio of interquartile range (IQR)/median value.

In the displaying of the graph, indicators respectively representing deviations of the plurality of elasticity values may further be displayed.

The selecting of at least one of the plurality of measurement values may include comparing each of the plurality of measurement values with the determined at least one reference value and selecting the at least one measurement value based on a result of the comparing.

The at least one reference value may include a first quartile and a third quartile for the plurality of elasticity values. The selecting of the at least one measurement value may include selecting at least one measurement value including an elasticity value that is greater than or equal to the first quartile and less than or equal to the third quartile.

The method may further include calculating reliability indices for the plurality of elasticity values, and the graph may further include the calculated reliability indices.

The selecting of the at least one measurement value may include receiving a user input for the plurality of measurement values and selecting the at least one measurement value and excluding some measurement values from the plurality of measurement values, based on the user input.

According to an aspect of another exemplary embodiment, an ultrasound diagnosis apparatus includes: a controller configured to acquire a plurality of pieces of elasticity data for an object and obtain a plurality of measurement values respectively from the plurality of pieces of elasticity data; and a display configured to display the plurality of measurement values, wherein the controller is further configured to select at least one of the plurality of measurement values, control the display to display the selected at least one measurement value to be distinguished from non-selected measurement values among the plurality of measurement values, and control a resultant value calculated based on the selected at least one measurement value to be displayed via the display.

The plurality of measurement values may include a plurality of elasticity values respectively acquired from the plurality of pieces of elasticity data. The controller may determine at least one reference value determined based on the plurality of elasticity values respectively acquired from the plurality of pieces of elasticity data. The display may display a graph representing the determined at least one reference value and the plurality of measurement values.

The at least one reference value may include at least one of a median value, a first quartile, and a third quartile for the plurality of elasticity values.

The displayed graph may include at least one baseline representing a magnitude of the at least one reference value.

The resultant value may include at least one of a median value, an average value, a reliability index, a standard deviation, and a ratio of IQR/median value.

The display may further display indicators respectively representing deviations of the plurality of elasticity values.

The controller may compare each of the plurality of measurement values with the determined at least one reference value and select the at least one measurement value based on a result of the comparing.

The at least one reference value may include a first quartile and a third quartile for the plurality of elasticity values, and the controller may select at least one measurement value including an elasticity value that is greater than or equal to the first quartile and less than or equal to the third quartile.

The controller may be further configured to calculate reliability indices for the plurality of elasticity values, and the graph displayed on the display may further include the calculated reliability indices.

The apparatus may further include an input device configured to receive a user input for the plurality of measurement values, and the controller may select the at least one measurement value and excludes some measurement values from the plurality of measurement values, based on the user input.

According to an aspect of another exemplary embodiment, a non-transitory computer-readable recording medium has recorded thereon a program for performing the above method.

### BRIEF DESCRIPTION OF THE DRAWINGS

Exemplary embodiments will now be described more fully hereinafter with reference to the accompanying drawings, in which reference numerals denote structural elements.

FIG. 1 is a block diagram of a configuration of an ultrasound diagnosis apparatus according to an exemplary embodiment;

FIG. 2 is a block diagram of a configuration of a wireless probe according to an exemplary embodiment;

FIG. 3 is a simplified block diagram of an ultrasound diagnosis apparatus according to an exemplary embodiment;

FIG. 4 is a flowchart of operations performed by an ultrasound diagnosis apparatus, according to an exemplary embodiment;

FIG. 5 is a diagram for explaining a shear wave;

FIG. 6 is a diagram for explaining a shear wave generated in an object;

FIG. 7 is a diagram for explaining a method of displaying an ultrasound image and elasticity, according to an exemplary embodiment;

FIGS. 8 and 9 illustrate user interfaces displayed according to an exemplary embodiment;

FIG. 10 illustrates a user interface displayed according to another exemplary embodiment;

FIGS. 11 through 13 illustrate a user interface that allows a user to select an elasticity value, according to an exemplary embodiment;

FIG. 14 illustrates a user interface that allows a user to select a measurement value by performing automatic examination, according to another exemplary embodiment;

FIGS. 15 and 16 illustrate a user interface that allows a user to select a measurement value according to another exemplary embodiment;

FIG. 17 illustrates a graph including information related to a measurement value, according to an exemplary embodiment;

FIGS. 18 through 20 illustrate a user interface for displaying information related to a measurement value, according to another exemplary embodiment;

FIGS. 21A and 21B are exemplary diagrams of an indicator included in a graph, according to an exemplary embodiment; and

FIG. 22 is a flowchart of operations performed by an ultrasound diagnosis apparatus according to another exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. In addition, parts not related to the present disclosure are omitted to clarify the description of exemplary embodiments. As used herein, the term “and/or” includes any and all combinations of one or more of the associated listed items. Expressions such as “at least one of,” when preceding a list of elements, modify the entire list of elements and do not modify the individual elements of the list.

The terms used in this specification are those general terms currently widely used in the art in consideration of functions regarding the inventive concept, but the terms may vary according to the intention of those of ordinary skill in the art, precedents, or new technology in the art. Also, some terms may be arbitrarily selected by the applicant, and in this case, the meaning of the selected terms will be described in detail in the detailed description of the present specification. Thus, the terms used herein have to be defined based on the meaning of the terms together with the description throughout the specification.

Throughout the specification, it will also be understood that when a component “includes” an element, unless there is another opposite description thereto, it should be understood that the component does not exclude another element and may further include another element.

In addition, terms such as “ . . . unit”, “ . . . module”, or the like refer to units that perform at least one function or operation, and the units may be implemented as hardware or software or as a combination of hardware and software.

Throughout the specification, an “ultrasound image” refers to an image of an object, which is obtained using ultrasound waves. Furthermore, an “object” may be a human, an animal, or a part of a human or animal. For example, the object may be an organ (e.g., the liver, the heart, the womb, the brain, a breast, or the abdomen), a blood vessel, or a combination thereof. Also, the object may be a phantom. The phantom means a material having a density, an effective atomic number, and a volume that are approximately the same as those of an organism. For example, the phantom may be a spherical phantom having properties similar to a human body.

Throughout the specification, a “user” may be, but is not limited to, a medical expert, for example, a medical doctor, a nurse, a medical laboratory technologist, or a medical imaging expert, or a technician who repairs medical apparatuses.

Hereinafter the inventive concept will be described in detail with reference to attached drawings.

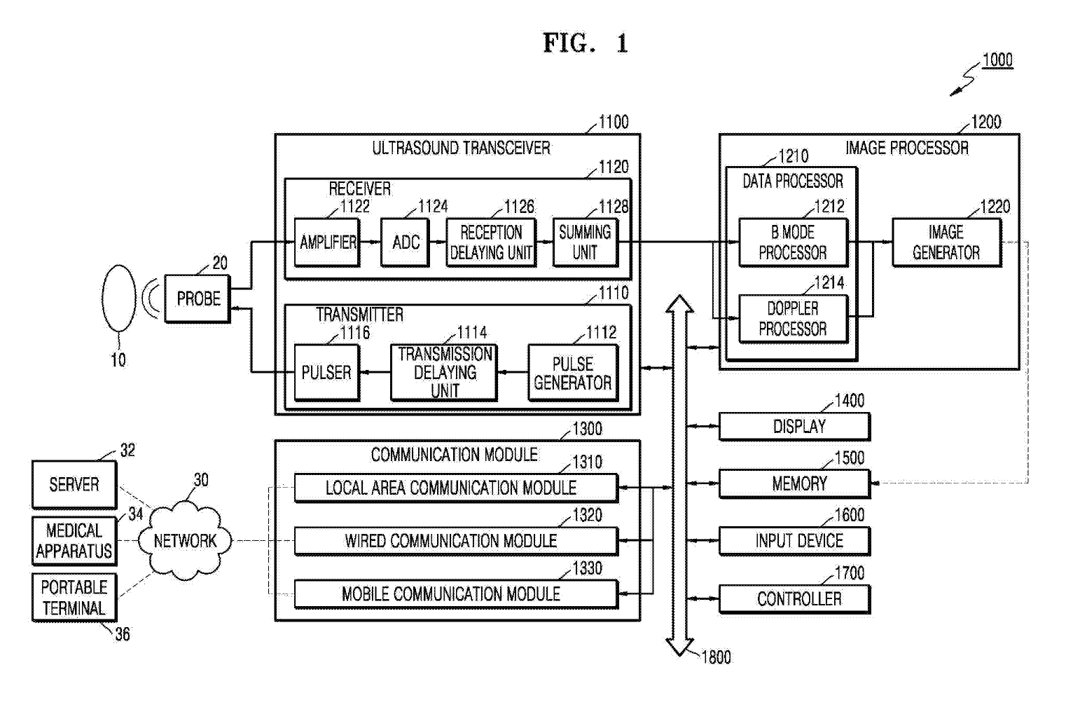

FIG. 1 is a block diagram showing a configuration of an ultrasound diagnosis apparatus 1000 according to an exemplary embodiment. Referring to FIG. 1, the ultrasound diagnosis apparatus 1000 may include a probe 20, an ultrasound transceiver 1100, an image processor 1200, a communication module 1300, a display 1400, a memory 1500, an input device 1600, and a controller 1700, which may be connected to one another via buses 1800.

The ultrasound diagnosis apparatus 1000 may be a cart type apparatus or a portable type apparatus. Examples of portable ultrasound diagnosis apparatuses may include, but are not limited to, a picture archiving and communication system (PACS) viewer, a smartphone, a laptop computer, a personal digital assistant (PDA), and a tablet PC.

The probe 20 transmits ultrasound waves to an object 10 in response to a driving signal applied by the ultrasound transceiver 1100 and receives echo signals reflected by the object 10. The probe 20 includes a plurality of transducers, and the plurality of transducers oscillate in response to electric signals and generate acoustic energy, that is, ultrasound waves. Furthermore, the probe 20 may be connected to the main body of the ultrasound diagnosis apparatus 1000 by wire or wirelessly, and according to exemplary embodiments, the ultrasound diagnosis apparatus 1000 may include a plurality of probes 20.

A transmitter 1110 supplies a driving signal to the probe 20. The transmitter 110 includes a pulse generator 1112, a transmission delaying unit 1114, and a pulser 1116. The pulse generator 1112 generates pulses for forming transmission ultrasound waves based on a predetermined pulse repetition frequency (PRF), and the transmission delaying unit 1114 delays the pulses by delay times necessary for determining transmission directionality. The pulses which have been delayed correspond to a plurality of piezoelectric vibrators included in the probe 20, respectively. The pulser 1116 applies a driving signal (or a driving pulse) to the probe 20 based on timing corresponding to each of the pulses which have been delayed.

A receiver 1120 generates ultrasound data by processing echo signals received from the probe 20. The receiver 120 may include an amplifier 1122, an analog-to-digital converter (ADC) 1124, a reception delaying unit 1126, and a summing unit 1128. The amplifier 1122 amplifies echo signals in each channel, and the ADC 1124 performs analog-to-digital conversion with respect to the amplified echo signals. The reception delaying unit 1126 delays digital echo signals output by the ADC 1124 by delay times necessary for determining reception directionality, and the summing unit 1128 generates ultrasound data by summing the echo signals processed by the reception delaying unit 1166. In some exemplary embodiments, the receiver 1120 may not include the amplifier 1122. In other words, if the sensitivity of the probe 20 or the capability of the ADC 1124 to process bits is enhanced, the amplifier 1122 may be omitted.

The image processor 1200 generates an ultrasound image by scan-converting ultrasound data generated by the ultrasound transceiver 1100. The ultrasound image may be not only a grayscale ultrasound image obtained by scanning an object in an amplitude (A) mode, a brightness (B) mode, and a motion (M) mode, but also a Doppler image showing a movement of an object via a Doppler effect. The Doppler image may be a blood flow Doppler image showing flow of blood (also referred to as a color Doppler image), a tissue Doppler image showing a movement of tissue, or a spectral Doppler image showing a moving speed of an object as a waveform.

A B mode processor 1212 extracts B mode components from ultrasound data and processes the B mode components. An image generator 1220 may generate an ultrasound image indicating signal intensities as brightness based on the extracted B mode components 1212.

Similarly, a Doppler processor 1214 may extract Doppler components from ultrasound data, and the image generator 1220 may generate a Doppler image indicating a movement of an object as colors or waveforms based on the extracted Doppler components.

According to an exemplary embodiment, the image generator 1220 may generate a three-dimensional (3D) ultrasound image via volume-rendering with respect to volume data and may also generate an elasticity image by imaging deformation of the object 10 due to pressure. Furthermore, the image generator 1220 may display various pieces of additional information in an ultrasound image by using text and graphics. In addition, the generated ultrasound image may be stored in the memory 1500.

A display 1400 displays the generated ultrasound image. The display 1400 may display not only an ultrasound image, but also various pieces of information processed by the ultrasound diagnosis apparatus 1000 on a screen image via a graphical user interface (GUI). In addition, the ultrasound diagnosis apparatus 1000 may include two or more displays 1400 according to exemplary embodiments.

The communication module 1300 is connected to a network 30 by wire or wirelessly to communicate with an external device or a server. The communication module 1300 may exchange data with a hospital server or another medical apparatus in a hospital, which is connected thereto via a PACS. Furthermore, the communication module 1300 may perform data communication according to the digital imaging and communications in medicine (DICOM) standard.

The communication module 1300 may transmit or receive data related to diagnosis of an object, e.g., an ultrasound image, ultrasound data, and Doppler data of the object, via the network 30 and may also transmit or receive medical images captured by another medical apparatus, e.g., a computed tomography (CT) apparatus, a magnetic resonance imaging (MRI) apparatus, or an X-ray apparatus. Furthermore, the communication module 1300 may receive information about a diagnosis history or medical treatment schedule of a patient from a server and utilizes the received information to diagnose the patient. Furthermore, the communication module 1300 may perform data communication not only with a server or a medical apparatus in a hospital, but also with a portable terminal of a medical doctor or patient.

The communication module 1300 is connected to the network 30 by wire or wirelessly to exchange data with a server 32, a medical apparatus 34, or a portable terminal 36. The communication module 1300 may include one or more components for communication with external devices. For example, the communication module 1300 may include a local area communication module 1310, a wired communication module 1320, and a mobile communication module 1330.

The local area communication module 1310 refers to a module for local area communication within a predetermined distance. Examples of local area communication techniques according to an exemplary embodiment may include, but are not limited to, wireless LAN, Wi-Fi, Bluetooth, ZigBee, Wi-Fi Direct (WFD), ultra wideband (UWB), infrared data association (IrDA), Bluetooth low energy (BLE), and near field communication (NFC).

The wired communication module 1320 refers to a module for communication using electric signals or optical signals. Examples of wired communication techniques according to an exemplary embodiment may include communication via a twisted pair cable, a coaxial cable, an optical fiber cable, and an Ethernet cable.

The mobile communication module 1330 transmits or receives wireless signals to or from at least one selected from a base station, an external terminal, and a server on a mobile communication network. The wireless signals may be voice call signals, video call signals, or various types of data for transmission and reception of text/multimedia messages.

The memory 1500 stores various data processed by the ultrasound diagnosis apparatus 1000. For example, the memory 1500 may store medical data related to diagnosis of an object, such as ultrasound data and an ultrasound image that are input or output, and may also store algorithms or programs which are to be executed in the ultrasound diagnosis apparatus 1000.

The memory 1500 may be any of various storage media, e.g., a flash memory, a hard disk drive, EEPROM, etc. Furthermore, the ultrasound diagnosis apparatus 1000 may utilize web storage or a cloud server that performs the storage function of the memory 1500 online.

The input device 1600 refers to a means via which a user inputs data for controlling the ultrasound diagnosis apparatus 1000. The input device 1600 may include hardware components, such as a keypad, a mouse, a touch pad, a touch screen, and a jog switch. However, exemplary embodiments are not limited thereto, and the input device 1600 may further include any of various other input units including an electrocardiogram (ECG) measuring module, a respiration measuring module, a voice recognition sensor, a gesture recognition sensor, a fingerprint recognition sensor, an iris recognition sensor, a depth sensor, a distance sensor, etc.

The controller 1700 may control all operations of the ultrasound diagnosis apparatus 1000. In other words, the controller 1700 may control operations among the probe 20, the ultrasound transceiver 1100, the image processor 1200, the communication module 1300, the display 1400, the memory 1500, and the input device 1600 shown in FIG. 1.

All or some of the probe 20, the ultrasound transceiver 1100, the image processor 1200, the communication module 1300, the display 1400, the memory 1500, the input device 1600, and the controller 1700 may be implemented as software modules. Also, at least one of the ultrasound transmission/reception unit 1100, the image processor 1200, and the communication module 1300 may be included in the control unit 1600; however, the inventive concept is not limited thereto.

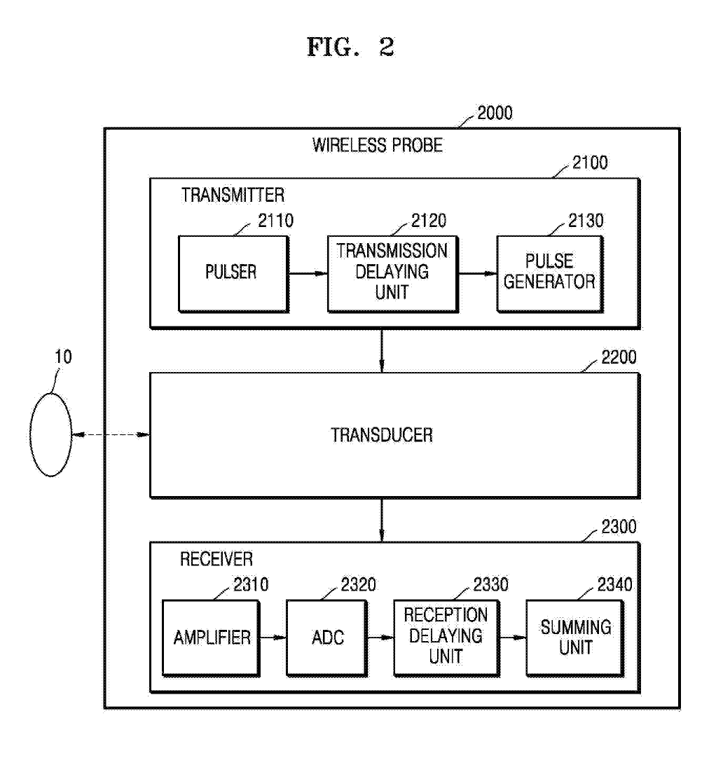

FIG. 2 is a block diagram showing a configuration of a wireless probe 2000 according to an exemplary embodiment. As described above with reference to FIG. 1, the wireless probe 2000 may include a plurality of transducers, and, according to exemplary embodiments, may include some or all of the components of the ultrasound transceiver 100 shown in FIG. 1.

The wireless probe 2000 according to the exemplary embodiment shown in FIG. 2 includes a transmitter 2100, a transducer 2200, and a receiver 2300. Since descriptions thereof are given above with reference to FIG. 1, detailed descriptions thereof will be omitted here. In addition, according to exemplary embodiments, the wireless probe 2000 may selectively include a reception delaying unit 2330 and a summing unit 2340.

The wireless probe 2000 may transmit ultrasound signals to the object 10, receive echo signals from the object 10, generate ultrasound data, and wirelessly transmit the ultrasound data to the ultrasound diagnosis apparatus 1000 shown in FIG. 1.

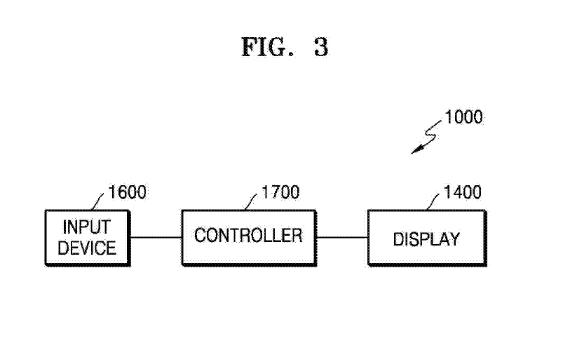

FIG. 3 is a simplified block diagram of a structure of the ultrasound diagnosis apparatus 1000 according to an exemplary embodiment.

The ultrasound diagnosis apparatus 1000 according to the present exemplary embodiment may include a controller 1700, a display 1400, and an input device 1600. However, according to an exemplary embodiment, the ultrasound diagnosis apparatus 1000 may include fewer components than those shown in FIG. 3. For example, the ultrasound diagnosis apparatus 1000 may not include the input device 1600 and receive a user input from a separate device.

According to an exemplary embodiment, the controller 1700 may acquire a plurality of pieces of elasticity data of an object. The controller 1700 may also acquire a plurality of measurement values from the plurality of pieces of elasticity data. A measurement value may refer to a value that may be acquired from elasticity data, such as a velocity of a transverse wave, an elasticity value, a reliability index for the elasticity value, or a standard deviation of elasticity values. In the specification, an ‘elasticity value’ may be replaced by another measurement value.

According to an exemplary embodiment, the controller 1700 may acquire a plurality of elasticity values of an object. A method of acquiring the plurality of elasticity values may be implemented in various ways according to exemplary embodiments. For example, if the user sets a region of interest (ROI) from an ultrasound image, the ultrasound diagnosis apparatus 1000 may obtain an elasticity value of an object by transmitting an ultrasound signal for pushing a region of the object via the ultrasound transceiver (1100 of FIG. 1) and tracking a shear wave produced by the transmitted ultrasound signal via the probe (20 of FIG. 1). The ultrasound diagnosis apparatus 1000 may obtain a plurality of elasticity values of the object by performing a process of acquiring an elasticity value of the object a plurality of times. As another example, the controller 1700 may obtain a plurality of elasticity values stored in the memory (1500 of FIG. 1) or acquire a plurality of elasticity values received from another device via the communication module (1300 of FIG. 1). However, exemplary embodiments are not limited thereto.

The controller 1700 may acquire at least one reference value for the acquired plurality of elasticity values. In this case, the reference value may be a value that may be used as a reference in determining whether elasticity values acquired by the user are appropriate values. According to an exemplary embodiment, the reference value may include at least one of a median, a first quartile, and a third quartile. The median refers to a middle value in acquired elasticity values when they are arranged in order of magnitude. The first quartile is a value that has 25 percent of acquired elasticity values below the value when the acquired elasticity values are arranged in order of magnitude. The third quartile is a value that has 75 percent of acquired elasticity values below the value when the acquired elasticity values are arranged in order of magnitude.

The controller 1700 may create a graph based on the acquired plurality of elasticity values and at least one reference value. In this case, the controller 1700 may generate a graph that allows a user to easily determine whether each of the acquired elasticity values is an appropriate value. According to an exemplary embodiment, the controller 1700 may create a graph that includes an indicator representing at least one baseline whose position is determined according to at least one reference value and an elasticity value. The user may determine whether an elasticity value acquired based on an indicator representing at least one baseline and an elasticity value is appropriate. For example, if an indicator representing an elasticity value in a graph is located on a plane lying above a baseline corresponding to the first quartile and below a baseline corresponding to the third quartile, the user may determine that the elasticity value is appropriate. According to another exemplary embodiment, the controller 1700 may control an indicator representing an elasticity value to have different shapes or colors depending on at least one reference value. For example, if an elasticity value is greater than the first quartile but less than the third quartile, the elasticity value may be indicated using a green indicator. Otherwise, the elasticity value may be indicated using a red indicator. Furthermore, the controller 1700 may calculate a deviation and a standard deviation for each of the acquired plurality of elasticity values. The controller 1700 may control an indicator representing a deviation or standard deviation of an elasticity value to be displayed via the display 1400 together with an indicator representing the elasticity value.

Furthermore, the controller 1700 may calculate a reliability index for each elasticity value. For example, the controller 1700 may compute a reliability index for an elasticity value based on a magnitude of a shear wave detected to obtain the elasticity value. In other words, the controller 1700 may determine that a reliability index for an elasticity value is high if a shear wave has a large magnitude and is low if the shear wave has a small magnitude. However, exemplary embodiments are not limited thereto. For example, the controller 1700 may determine a reliability index of an elasticity value based on a residual of a shear wave as well as a magnitude of the shear wave. In other words, the controller 1700 may determine that a reliability index of an elasticity value is high if a shear wave has a small residual, or is low if the shear wave has a large residual.

The controller 1700 may select some of a plurality of elasticity values. A method of selecting some of a plurality of elasticity values may be implemented in various ways according to exemplary embodiments. According to an exemplary embodiment, the controller 1700 may select at least one of a plurality of elasticity values based on a user input received via the input device 1600. For example, if the user selects an elasticity value that is determined to be inappropriate via the input device 1600, the controller 1700 may select an elasticity value, other than the selected elasticity value, from among the plurality of elasticity values. Alternatively, the controller 1700 may select an elasticity value selected by the user. According to anther exemplary embodiment, the controller 1700 may select at least one of the plurality of elasticity values based on at least one reference value. For example, if the at least one reference value includes first and third quartiles, the controller 1700 may select an elasticity value that is greater than or equal to the first quartile and less than or equal to the third quartile. According to another exemplary embodiment, the controller 1700 may select at least one elasticity value having a reliability index higher than or equal to a threshold value, based on a reliability index for an elasticity value. In this case, the threshold value may be a predetermined value or may be preset based on a user input.

The controller 1700 may compute a resultant value based on a selected at least one elasticity value. According to an exemplary embodiment, the resultant value may include a median value, an average value, and a satisfaction level for the selected at least one elasticity value. A satisfaction level may be defined as a value indicating a quality for a median or average value of a selected at least one elasticity value, and may be computed in diverse ways according to exemplary embodiments. According to an exemplary embodiment, the satisfaction level may be obtained by dividing an interquartile range (IQR) by a median value. In this case, an IQR may be a distance between the first and third quartiles divided by 2.

The display 1400 may display a graph generated by the controller 1700. The graph displayed on the display 1400 may be changed in various ways according to exemplary embodiments. For example, the display 1400 may display a graph where an elasticity value is indicated by a dot, a broken line, a bar, or the like. Furthermore, the graph displayed on the display 1400 may include a baseline whose position is determined according to a reference value acquired by the controller 1700. In other words, the graph may include at least one baseline located so as to represent a magnitude of a reference value. Furthermore, the display 1400 may display reliability indices respectively assigned to a plurality of elasticity values.

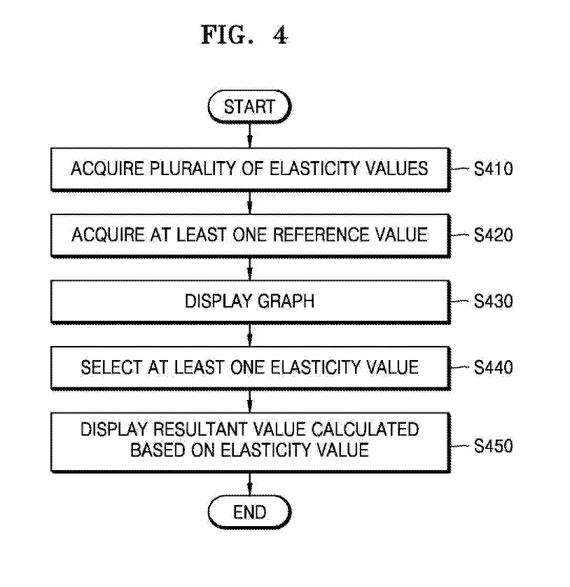

FIG. 4 is a flowchart of a process of performing operations performed by the ultrasound diagnosis apparatus (1000 of FIG. 1) according to an exemplary embodiment.

The ultrasound diagnosis apparatus 1000 may acquire a plurality of elasticity values for an object. A method of acquiring the plurality of elasticity values may be implemented in various ways according to exemplary embodiments. For example, if a user sets an ROI from an ultrasound image, the ultrasound diagnosis apparatus 1000 may obtain an elasticity value of the object by transmitting an ultrasound signal for pushing a region of the object to the object. The ultrasound diagnosis apparatus 1000 may also obtain an elasticity value by tracking a shear wave produced by the transmitted ultrasound signal. The ultrasound diagnosis apparatus 1000 may obtain a plurality of elasticity values of the object by performing a process of acquiring an elasticity value of the object a plurality of times. As another example, the controller 1700 may obtain a plurality of elasticity values stored in the memory 1500. Alternatively, the ultrasound diagnosis apparatus 1000 may acquire a plurality of elasticity values received from another device via the communication module 1300. However, exemplary embodiments are not limited thereto.

The ultrasound diagnosis apparatus 1000 may acquire at least one reference value for the acquired plurality of elasticity values (S420). According to an exemplary embodiment, a reference value may include at least one of a median, a first quartile, and a third quartile. The median refers to a middle value in acquired elasticity values when they are arranged in order of magnitude. The first quartile is a value that has 25 percent of acquired elasticity values below the value when the acquired elasticity values are arranged in order of magnitude. The third quartile is a value that has 75 percent of acquired elasticity values below the value when the acquired elasticity values are arranged in order of magnitude.

Thereafter, the ultrasound diagnosis apparatus 1000 may generate a graph based on the acquired plurality of elasticity values and at least one reference value (S430). Furthermore, the ultrasound diagnosis apparatus 1000 may display the generated graph via the display 1400. In this case, the ultrasound diagnosis apparatus 1000 may generate a graph that allows the user to easily determine whether each of the acquired elasticity values is an appropriate value. The graph displayed via the display 1400 may include an indicator representing at least one baseline whose position is determined according to at least one reference value and an elasticity value. According to another exemplary embodiment, the ultrasound diagnosis apparatus 1000 may control an indicator representing an elasticity value to have different shapes or colors depending on the at least one reference value. For example, if an elasticity value is greater than the first quartile but less than the third quartile, the elasticity value may be indicated using a green indicator. Otherwise, the elasticity value may be indicated using a red indicator. Furthermore, the ultrasound diagnosis apparatus 1000 may calculate a deviation and a standard deviation for each of the acquired plurality of elasticity values. The ultrasound diagnosis apparatus 1000 may control an indicator representing a deviation or standard deviation of an elasticity value to be displayed via the display 1400 together with an indicator representing the elasticity value.

Furthermore, after operation S410, the ultrasound diagnosis apparatus 1000 may calculate a reliability index for each of the plurality of elasticity values acquired in operation S410. For example, the ultrasound diagnosis apparatus 1000 may compute a reliability index for an elasticity value based on a magnitude of a shear wave detected to obtain the elasticity value. In other words, the ultrasound diagnosis apparatus 1000 may determine that a reliability index for an elasticity value is high if a shear wave has a large magnitude and is low if the shear wave has a small magnitude. However, exemplary embodiments are not limited thereto. For example, the ultrasound diagnosis apparatus 1000 may determine a reliability index of an elasticity value based on a residual of a shear wave as well as a magnitude of the shear wave. In other words, the ultrasound diagnosis apparatus 1000 may determine that a reliability index of an elasticity value is high if a shear wave has a small residual, or is low if the shear wave has a large residual.

The ultrasound diagnosis apparatus 1000 may select at least one of a plurality of elasticity values (S440). A method of selecting some of a plurality of elasticity values may be implemented in various ways according to exemplary embodiments. According to an exemplary embodiment, the ultrasound diagnosis apparatus 1000 may select at least one of the plurality of elasticity values from the graph displayed in operation S430. In this case, the ultrasound diagnosis apparatus 1000 may select at least one of the plurality of elasticity values based on a user input received via the input device 1600. For example, if the user selects an elasticity value that is determined to be inappropriate via the input device 1600, the ultrasound diagnosis apparatus 1000 may select an elasticity value, other than the selected elasticity value, from among the plurality of elasticity values. Alternatively, the ultrasound diagnosis apparatus 1000 may select an elasticity value selected by the user. According to anther exemplary embodiment, the ultrasound diagnosis apparatus 1000 may select at least one of the plurality of elasticity values based on at least one reference value. For example, if the at least one reference value includes first and third quartiles, the ultrasound diagnosis apparatus 1000 may select an elasticity value that is greater than or equal to the first quartile and less than or equal to the third quartile. According to another exemplary embodiment, the ultrasound diagnosis apparatus 1000 may select at least one elasticity value having a reliability index greater than or equal to a threshold value, based on a reliability index for an elasticity value. In this case, the threshold value may be a predetermined value or may be preset based on a user input.

The ultrasound diagnosis apparatus 1000 may calculate a resultant value based on the selected at least one elasticity value (S450). Furthermore, the ultrasound diagnosis apparatus 1000 may display the calculated resultant value together with the graph displayed in operation S430. According to an exemplary embodiment, the resultant value may include a median value, an average value, and a satisfaction level of the selected at least one elasticity value. The satisfaction level may be defined as a value indicating a quality for the median or average value of the selected at least one elasticity value, and may be computed in diverse ways according to exemplary embodiments. According to an exemplary embodiment, the satisfaction level may be obtained by dividing an interquartile range (IQR) by a median value. In this case, an IQR may be a distance between the first and third quartiles divided by 2.

The graph displayed on the display 1400 may be changed in various ways according to exemplary embodiments. For example, the display 1400 may display a graph where an elasticity value is indicated by a point, broken line, bar, or the like. Furthermore, the graph displayed on the display 1400 may include a baseline whose position is determined according to a reference value acquired by the controller 1700. In other words, the graph may include at least one baseline located so as to represent a magnitude of a reference value. Furthermore, the display 1400 may display reliability indices respectively assigned to a plurality of elasticity values.

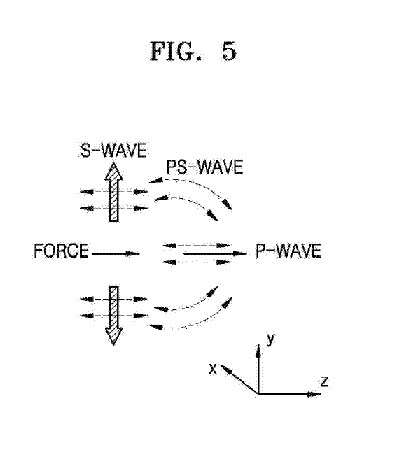

FIG. 5 is a diagram for explaining a shear wave.

Referring to FIG. 5, when a force corresponding to a point impulse is applied in a Z-axis direction, a p-wave that is a longitudinal wave, an s-wave that is a transverse wave, and a ps-wave consisting of the p- and s-waves coupled together are generated. In this case, the shear wave is the s-wave that vibrates in a direction of wave propagation from a vibrating source to which the force is applied and propagates in a Y-axis direction.

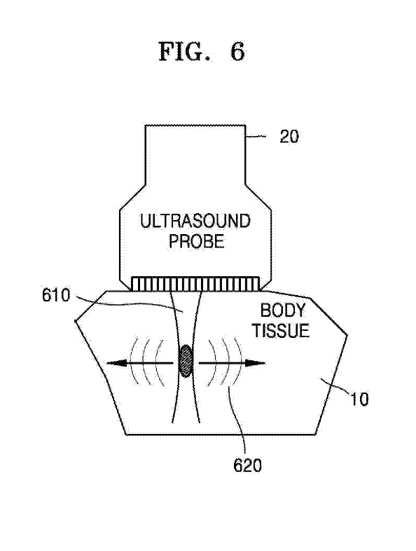

FIG. 6 is a diagram for explaining a shear wave generated in an object 10.

Referring to FIG. 6, the ultrasound diagnosis apparatus 1000 may transmit an ultrasound signal 610 for pushing the object 10 (for convenience of explanation, hereinafter referred to as a ‘push ultrasound signal’) to the object 10. For example, the ultrasound diagnosis apparatus 1000 may transmit the push ultrasound signal 610 of a long wavelength to the object 10 via some of channels of a probe 20. According to an exemplary embodiment, the ultrasound diagnosis apparatus 1000 may transmit the focused push ultrasound signal 610 to a region of the object 10.

In this case, a shear wave 620 may be generated in the object 10 by the push ultrasound signal 610. For example, the shear wave 620 may be generated around a region pushed by the push ultrasound signal 610. The shear wave 620 may travel at a velocity of about 1 m/s to about 10 m/s. Since the velocity of the shear wave 620 is still less than an average velocity (about 1540 m/s) of an ultrasound signal in the object 10, the ultrasound diagnosis apparatus 1000 may use an ultrasound signal (hereinafter, referred to as a tracing ultrasound signal) for tracing the shear wave 620. For example, the ultrasound diagnosis apparatus 1000 may transmit the tracing ultrasound signal in a direction of propagation of the shear wave 620, thereby tracking the velocity of the shear wave 620. In this case, the tracing ultrasound signal may have a shorter wavelength than that of the push ultrasound signal 610.

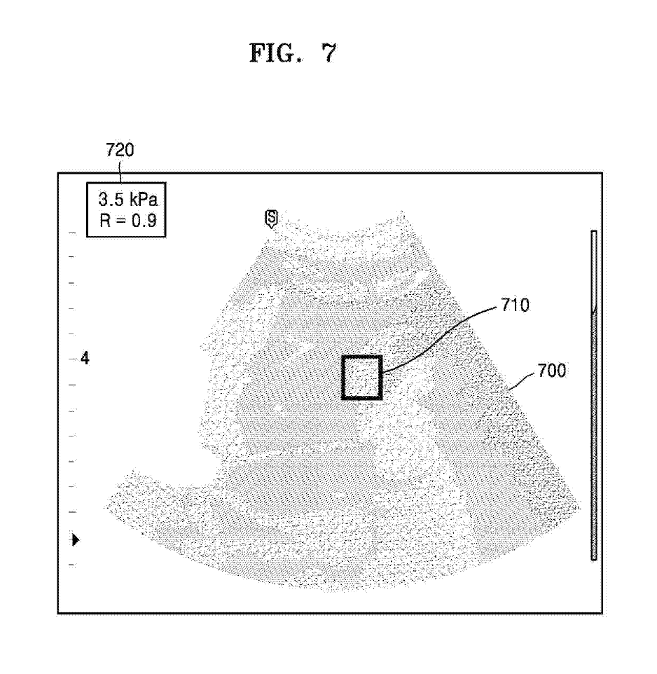

FIG. 7 is a diagram for explaining a method of displaying an ultrasound image 700 and elasticity according to an exemplary embodiment.

According to an exemplary embodiment, the ultrasound diagnosis apparatus (1000 of FIG. 1) according to an exemplary embodiment may acquire the ultrasound image 700 and display the acquired ultrasound image 700. In this case, if a user sets an ROI 710 from the ultrasound image 700 via the input device 1600, the ultrasound diagnosis apparatus 1000 may acquire an elasticity value of an object for the set ROI 710. The ultrasound diagnosis apparatus 1000 may then display the acquired elasticity value. Furthermore, the ultrasound diagnosis apparatus 1000 may calculate a reliability index for the acquired elasticity value and display the calculated reliability index together with the elasticity value. For example, as shown in 720 of FIG. 7, the ultrasound diagnosis apparatus 1000 may display an elasticity value and a reliability index. As shown in FIG. 7, the ultrasound diagnosis apparatus 1000 may obtain a plurality of elasticity values by performing a process of setting the ROI 710 and acquiring an elasticity value for the set ROI 710 a plurality of times.

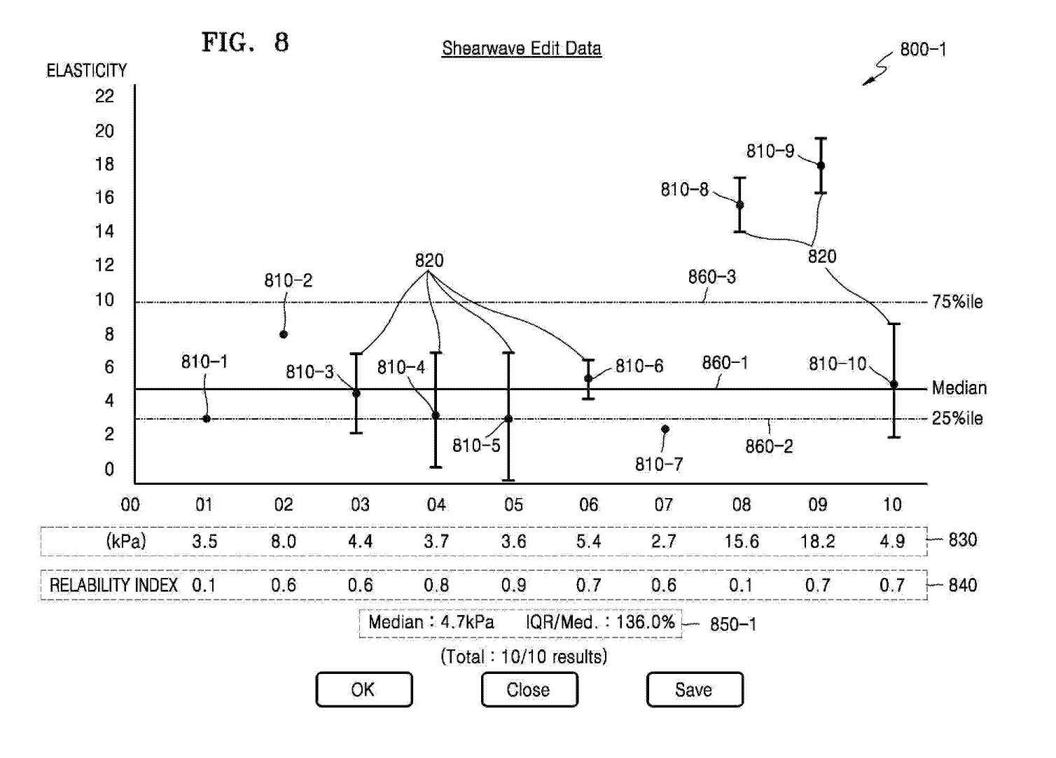

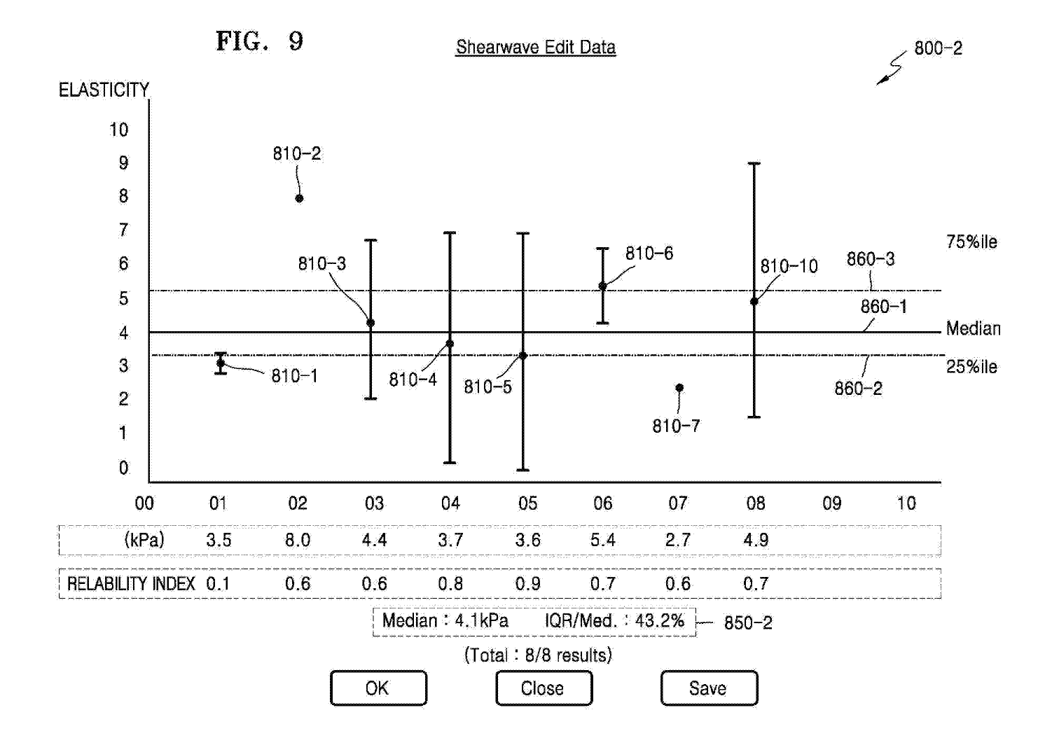

FIGS. 8 and 9 illustrate user interfaces displayed according to an exemplary embodiment.

The ultrasound diagnosis apparatus (1000 of FIG. 1) may display a graph 800-1 shown in FIG. 8 by performing the process illustrated in FIG. 4. The graph 800-1 may include indicators 810-1 through 810-10 representing elasticity values, indicators 820 representing deviations of the elasticity values represented by the indicators 810-3 through 810-6 and 810-10, and baselines 860-1 through 860-3 respectively representing a median, a first quartile (25% ile), and a third quartile (75% ile). By analyzing the graph 800-1, a user may easily determine whether an elasticity value is appropriate based on whether each of the indicators 810-1 through 810-10 representing its corresponding elasticity value is located between the baselines 860-2 and 860-3 respectively representing the first and third quartiles. Positions of the baselines 860-1 through 860-3 in the graph 800-1 may be determined according to magnitudes of reference values calculated based on the plurality of elasticity values. Furthermore, as seen in 830 and 840 of FIG. 8, the ultrasound diagnosis apparatus 1000 may indicate elasticity values and reliability indices for the elasticity values by numerical values. The ultrasound diagnosis apparatus 1000 may further indicate a resultant value 850-1 for the elasticity values. According to an exemplary embodiment, as shown in FIG. 8, a median and a ratio of IQR/median may be indicated as the resultant value 850-1. By analyzing the graph 800-1, the user may determine that at least one indicator (e.g., indicators 810-8 and 810-9) corresponding to elasticity values may represent inappropriate values. The user may select the at least one indicator (e.g., 810-8 and 810-9), which are determined to represent inappropriate values, via the input device 1600.

If the user selects the at least one indicator 810-8 and 810-9, as shown in FIG. 9, the ultrasound diagnosis apparatus 1000 may remove the selected at least one indicator 810-8 and 810-9 from the graph 800-1. Furthermore, the ultrasound diagnosis apparatus 1000 may update the reference values based on the remaining indicators 810-1 through 810-7 and 810-10 and display baselines 860-1 through 860-3 according to the updated reference values. The ultrasound diagnosis apparatus 1000 may also display a resultant value 850-2 newly calculated based on the remaining indicators 810-1 through 810-7 and 810-10. Furthermore, as the indicators 810-8 and 810-9 are excluded, the ultrasound diagnosis apparatus 1000 may change scales on an abscissa and/or ordinate of a graph 800-2.

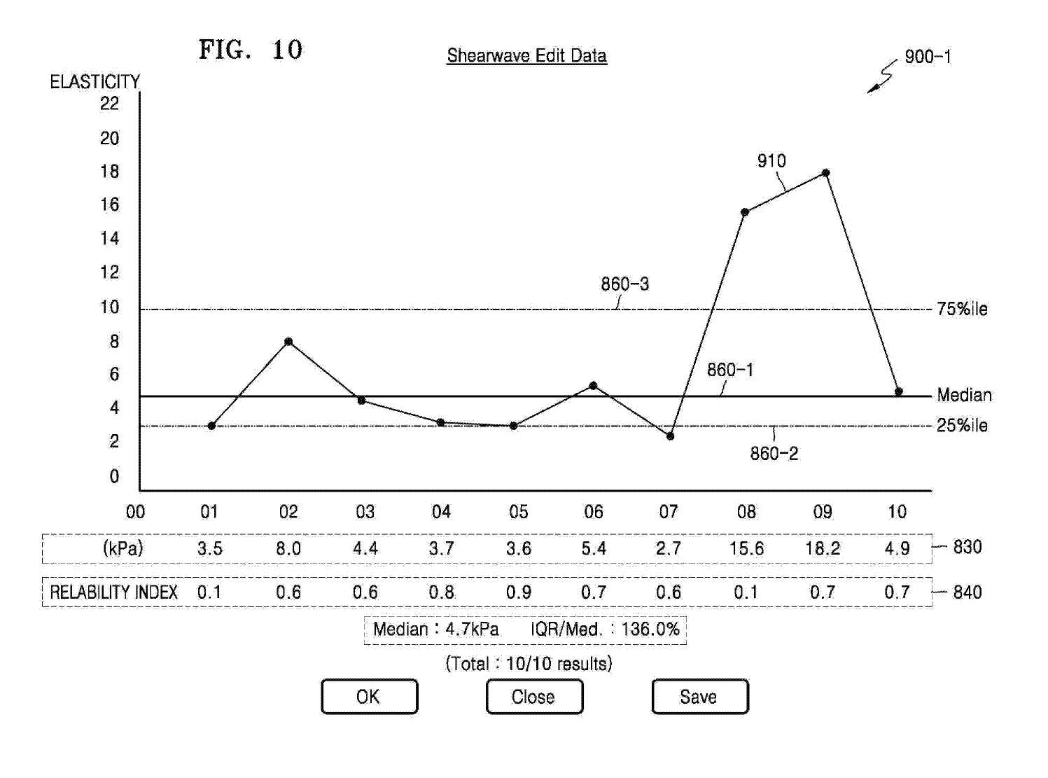

FIG. 10 illustrates a user interface displayed according to another exemplary embodiment.

Although FIGS. 8 and 9 show that elasticity values are indicated by points, the elasticity values may be indicated in various ways. Referring to FIG. 10, a plurality of elasticity values may be represented using a graph 900-1 including a broken line 910 and reference lines 860-1 through 860-3.

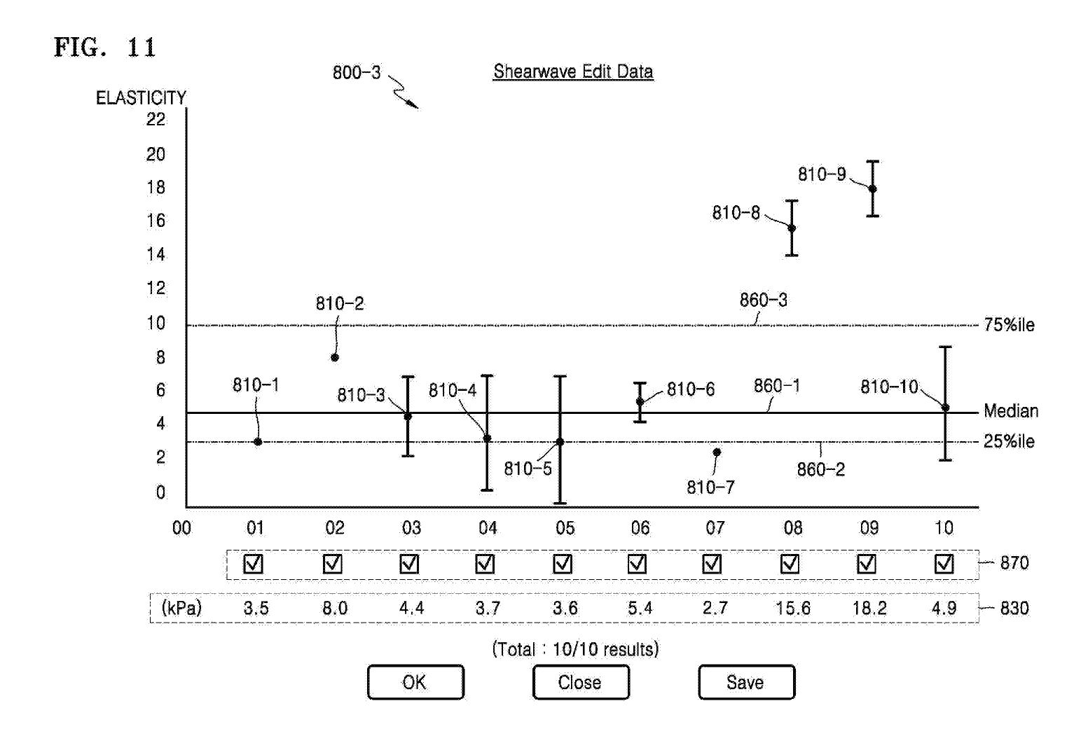

FIGS. 11 through 13 illustrate a user interface that allows a user to select an elasticity value according to another exemplary embodiment.

Referring to FIG. 11, the ultrasound diagnosis apparatus 1000 may display near a graph 800-3 interface elements (e.g., check boxes) 870 respectively corresponding to indicators 810-1 through 810-10 representing their corresponding elasticity values. By selecting the user interface elements (e.g., check boxes) 870 after examining the indicators 810-1 through 810-10 representing the elasticity values and baselines 860-1 through 860-3, the user may select or exclude elasticity values to be used for calculating a resultant value.

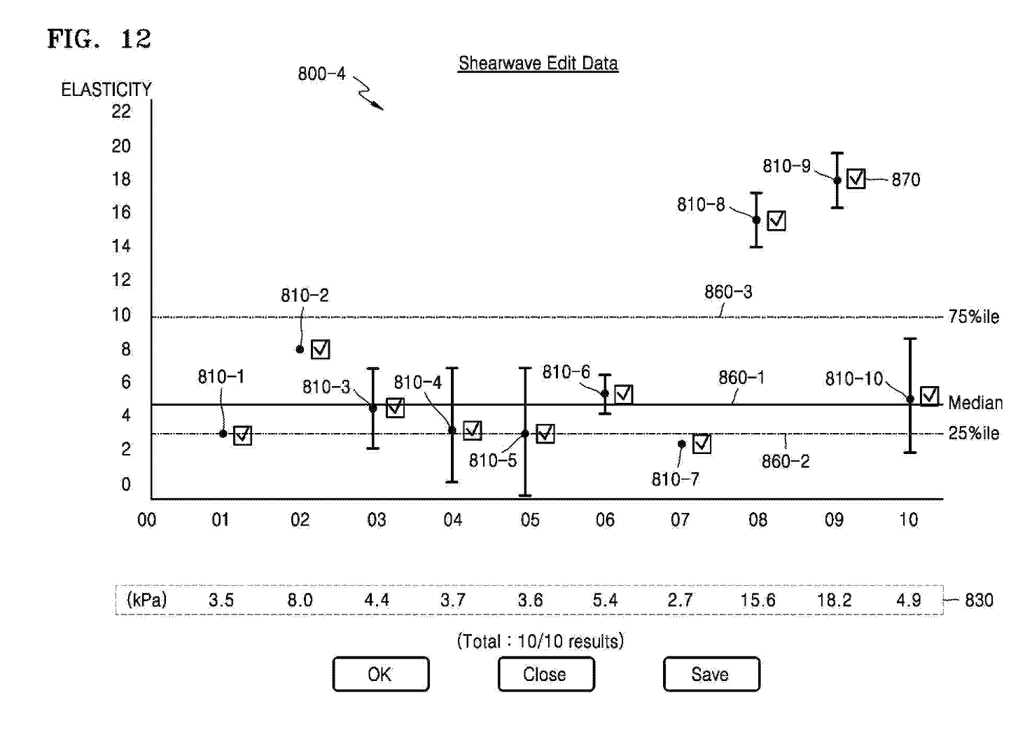

Alternatively, referring to FIG. 12, the ultrasound diagnosis apparatus 1000 may display a graph 800-4 where user interface elements (e.g., check boxes) 870 are respectively positioned next to indicators 810-1 through 810-10 representing elasticity values.

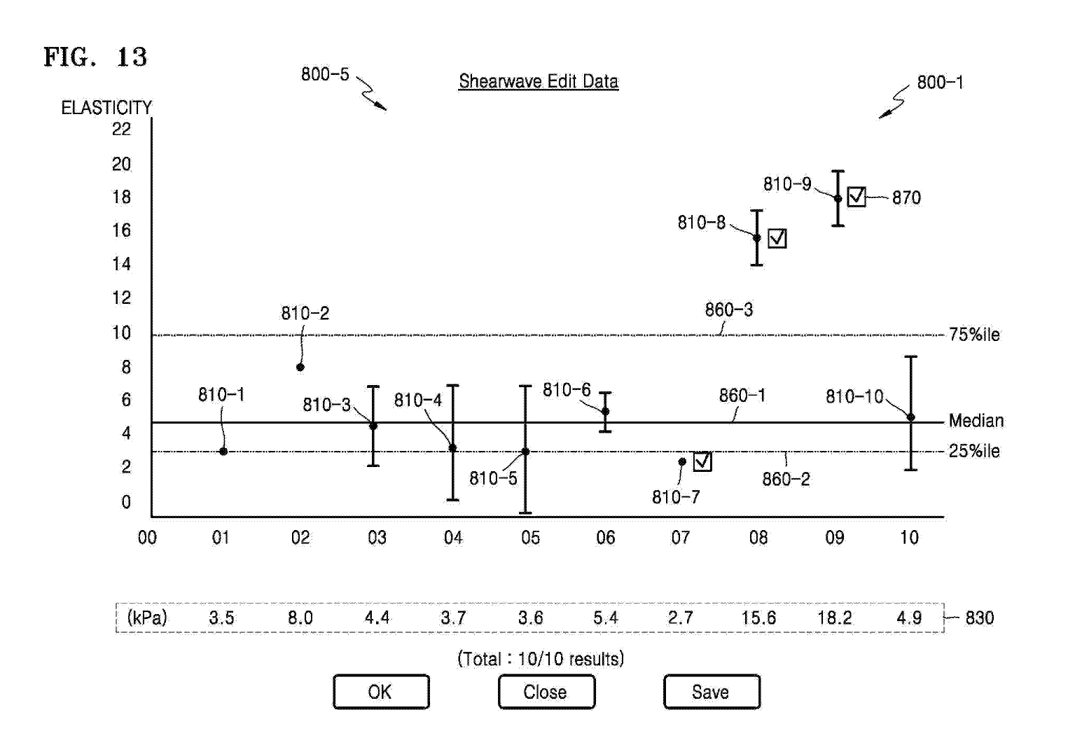

Furthermore, the ultrasound diagnosis apparatus 1000 may display only user interface elements (e.g., check boxes) 870 respectively corresponding to some of a plurality of elasticity values. For example, referring to FIG. 13, the ultrasound diagnosis apparatus 1000 may display a graph 800-5 including user interface elements for indicators (e.g., 810-7 through 810-9) representing elasticity values that are less than a first quartile or greater than a third quartile.

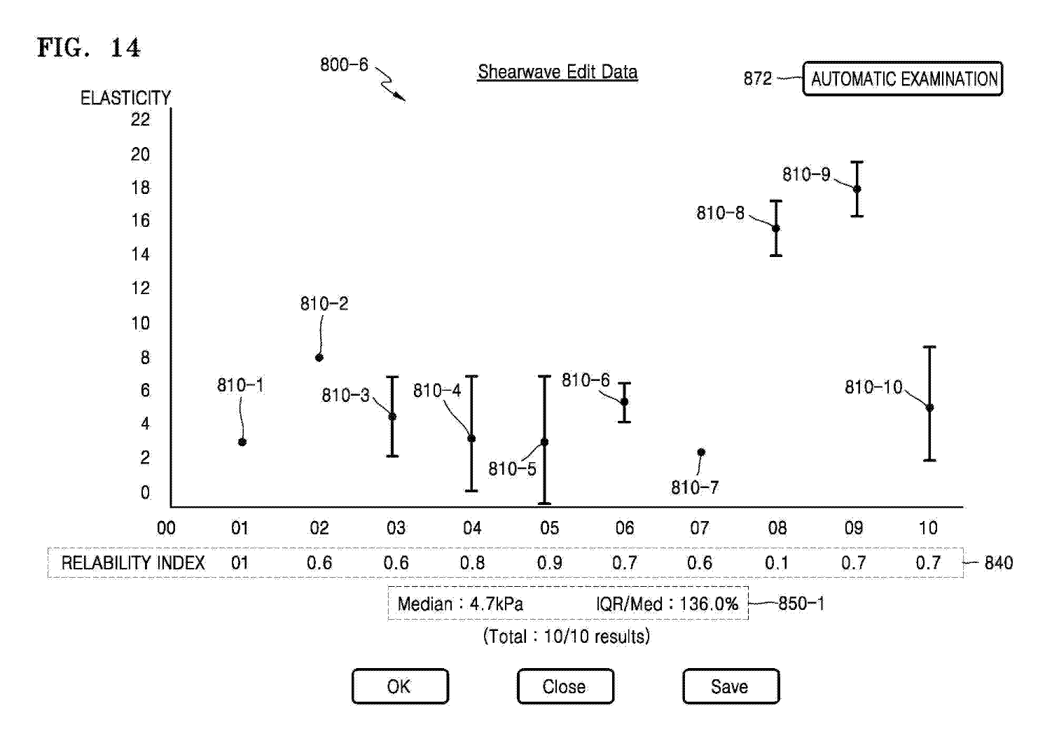

FIG. 14 illustrates a user interface that allows a user to select a measurement value by using an automatic examination, according to another exemplary embodiment

Referring to FIG. 14, the display 1400 may display a graph 800-6 including a user interface object (e.g., a button) 872 for automatically selecting indicators 810-1 through 810-10 respectively representing a plurality of measurement values (e.g., elasticity values) and the measurement values.

When the user selects the user interface object 872 for automatically selecting a measurement value, the controller 1700 of the ultrasound diagnosis apparatus 1000 may compare each of the plurality of measurement values with at least one reference value. The controller 1700 may select at least one of the measurement values based on a comparison result. Referring to FIG. 14, for example, the controller 1700 may compare each of a plurality of elasticity values with first and third quartiles for the plurality of elasticity values. Furthermore, the controller 1700 may select at least one elasticity value that is greater than or equal to the first quartile and less than or equal to the third quartile. The controller 1700 may also obtain a resultant value 850-1 based on the selected at least one elasticity value. The display 1400 may change the displayed resultant value 850-1 to a resultant value acquired based on the selected at least one elasticity value.

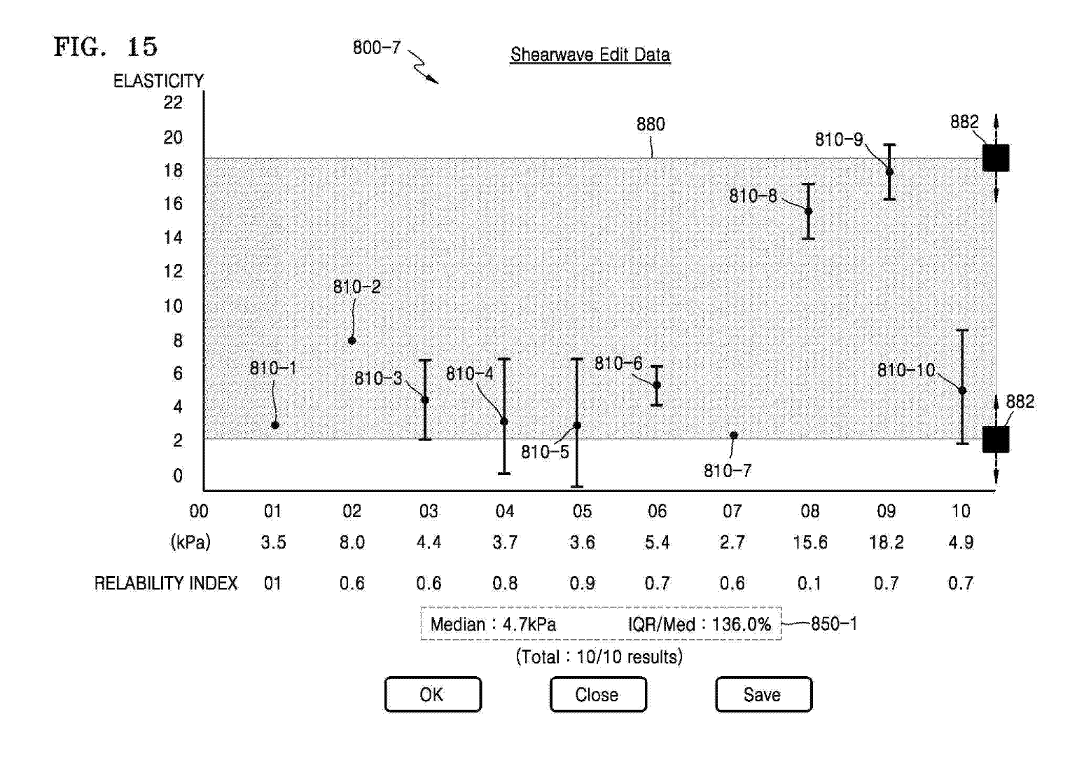

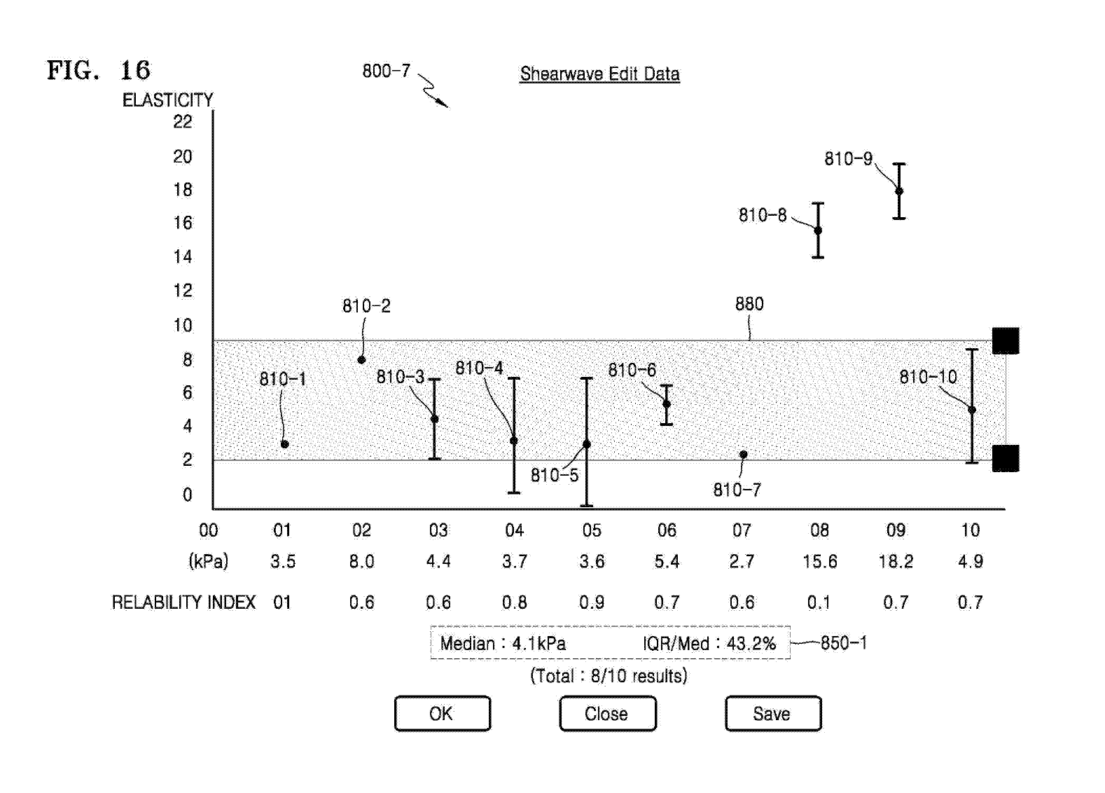

FIGS. 15 and 16 illustrate a user interface that allows a user to select a measurement value according to another exemplary embodiment.

Referring to FIG. 15, the display 1400 may display a graph 800-7 including a region 880 for selecting a measurement value (e.g., an elasticity value). The display 1400 may further display a user interface object 882 for adjusting the region 880. The user may use the user interface object 882 to control the region 880. For example, the user may adjust an area of the region 880 by dragging the user interface object 882.

The controller 1700 may select at least one measurement value from among a plurality of measurement values respectively represented by indicators 810-1 through 810-10. The controller 1700 may also obtain a resultant value based on the selected at least one measurement value. The display 1400 may change a displayed resultant value 850-1 to a resultant value acquired based on the selected at least one measurement value. For example, if an area of the region 880 is changed as shown in FIG. 16 by using the user interface object 882 included in the graph 800-7 of FIG. 15, the controller 1700 may select the plurality of measurement values 810-1 through 810-6 and 810-10 included in the region 880. In this case, the controller 1700 may acquire a resultant value 850-1 based on the selected measurement values 810-1 through 810-6 and 810-10. Thus, the display 1400 may change the displayed resultant value 850-1 to the resultant value acquired based on the selected at least one measurement value.

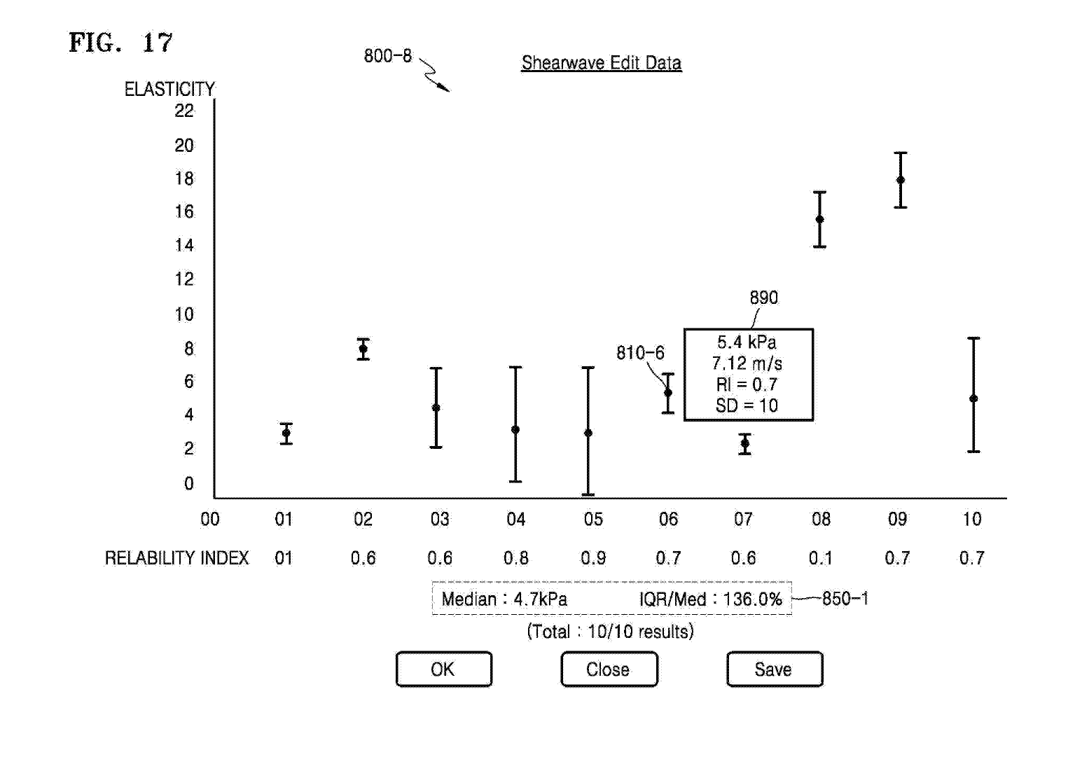

FIG. 17 illustrates a graph including information related to a measurement value, according to an exemplary embodiment.

According to an exemplary embodiment, one of a plurality of elasticity values (e.g., an elasticity value represented by an indicator 810-6) included in a graph 800-8 may be selected based on a user input received via the input device 1600. The display 1400 may display a measurement value 890 related to the selected elasticity value. For example, when the indicator 810-6 is selected, the display 1400 may display near the indicator 810-6 the measurement value 890 including at least one of an elasticity value, a velocity of a transverse wave, a reliability index for the elasticity value, and a standard deviation related to the elasticity value.

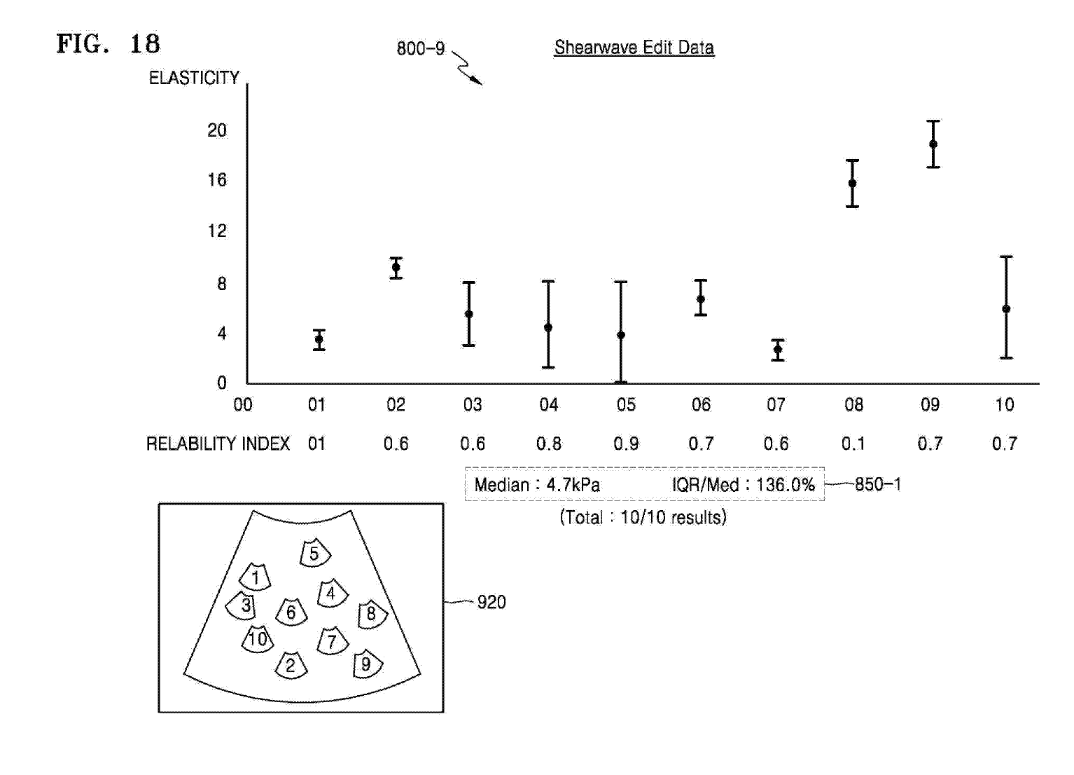

FIGS. 18 through 20 illustrate a user interface for displaying information related to a measurement value, according to another exemplary embodiment.

The display 1400 may further display an ROI related to a measurement value included in a graph 800-9. Referring to FIG. 18, the display 1400 may further display an image 920 indicating thereon positions of ROls respectively corresponding to a plurality of measurement values.

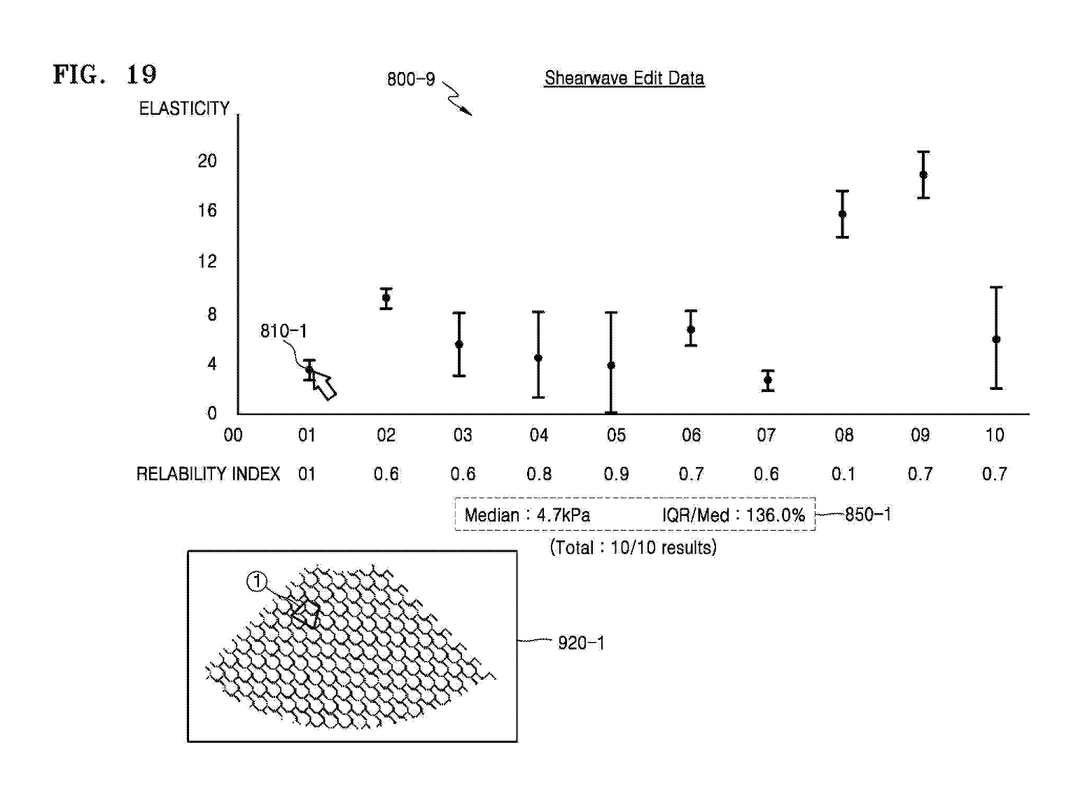

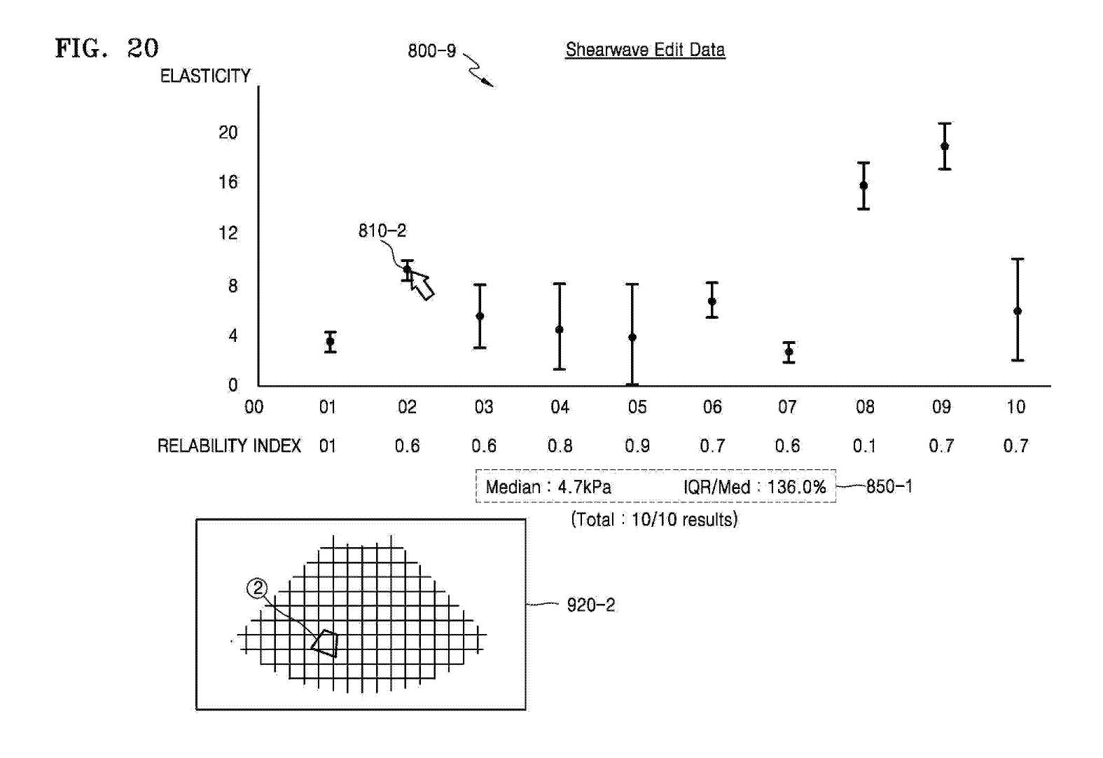

Furthermore, the display 1400 may display an ultrasound image corresponding to a measurement value selected from among a plurality of measurement values and a position of an ROI set in the ultrasound image. Referring to FIG. 19, if an indicator 810-1 is selected based on a user input received via the input device 1600, the display 1400 may display an ultrasound image 920-1 in which an ROI corresponding to the selected indicator 810-1 (i.e., a selected elasticity value) has been set. Furthermore, referring to FIG. 20, if an indicator 810-2 is selected based on a user input received via the input device 1600, the display 1400 may display an ultrasound image 920-2 in which an ROI corresponding to the selected indicator 810-2 has been set.

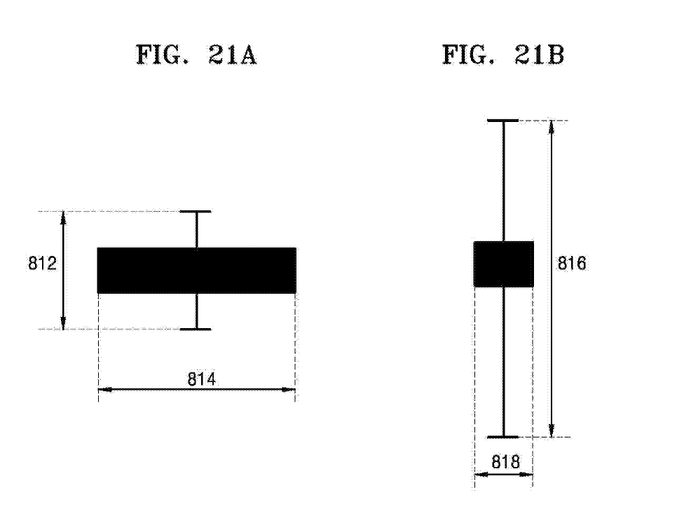

The display 1400 may display a measurement value in various ways according to exemplary embodiments. For example, a measurement value may be displayed using a width or area of an indicator. FIGS. 21A and 21B are exemplary diagrams of an indicator included in a graph according to an exemplary embodiment. For example, a deviation of an elasticity value may be indicated by a height of a marker, and a reliability index of the elasticity value may be represented by a width of a rectangle. In other words, to indicate an elasticity value having a small deviation and a high reliability index, the display 1400 may display an indicator including a marker 812 having a small height and a rectangle 814 having a large width as shown in FIG. 21A. On the other hand, to indicate an elasticity value having a large deviation and a low reliability index, the display 1400 may display an indicator including a marker 816 having a large height and a rectangle 818 having a small width as shown in FIG. 21B.

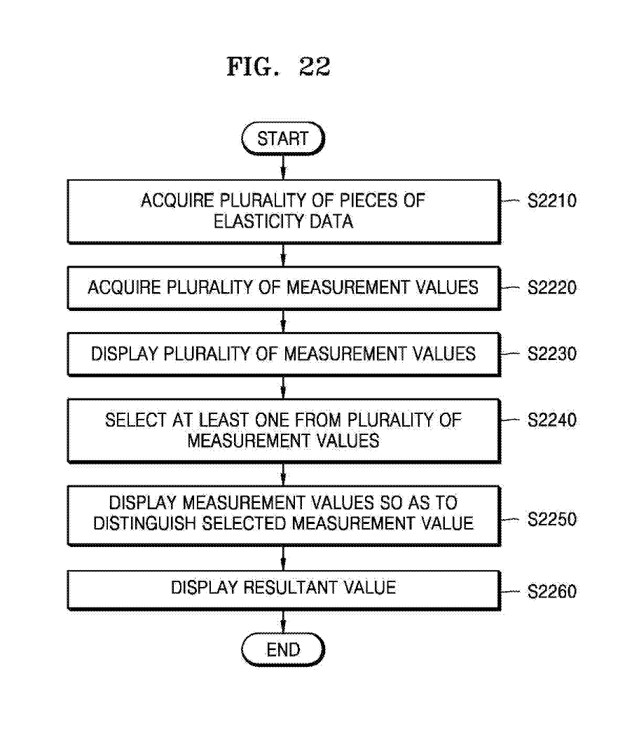

FIG. 22 is a flowchart of a process of performing operations of the ultrasound diagnosis apparatus (1000 of FIG. 1) according to another exemplary embodiment.

The ultrasound diagnosis apparatus 1000 may acquire a plurality of pieces of elasticity data (S2210). For example, the ultrasound diagnosis apparatus 1000 may acquire a plurality of pieces of elasticity data by performing a process of inducing a shear wave in an object and observing the induced shear wave via the probe 20 a plurality of times. Alternatively, the ultrasound diagnosis apparatus 1000 may receive a plurality of pieces of elasticity data from another device or acquire a plurality of pieces of elasticity data stored in the memory 1500.

Thereafter, the ultrasound diagnosis apparatus 1000 may acquire a plurality of measurement values from each of the plurality of pieces of elasticity data (S2220). In this case, the plurality of measurement values may include at least one of an elasticity value, a velocity of an observed transverse wave, a reliability index, and a deviation.

Subsequently, the ultrasound diagnosis apparatus 1000 may display the acquired plurality of measurement values (S2230). In operation S2230, the ultrasound diagnosis apparatus 1000 may generate a graph representing the acquired measurement values and display the generated graph.

The ultrasound diagnosis apparatus 1000 may select at least one of the plurality of measurement values (S2240). In operation S2240, the ultrasound diagnosis apparatus 1000 may select a measurement value based on a user input received via the input device 1600. Alternatively, the ultrasound diagnosis apparatus 1000 may compare each of the plurality of measurement values with reference values (e.g., a median, a first quartile, a third quartile for an elasticity value, etc.) determined based on the plurality of measurement values and select a measurement value based on a comparison result.

The ultrasound diagnosis apparatus 1000 may display measurement values in such a manner as to distinguish the selected at least one measurement value from non-selected measurement values (S2250). For example, as shown in FIG. 9, the ultrasound diagnosis apparatus 1000 may display only the selected at least one measurement value and exclude the non-selected measurement values. As another example, as shown in FIGS. 11 through 13, the ultrasound diagnosis apparatus 1000 may indicate whether a measurement value has been selected by using a check box.

As another example, as shown in FIGS. 15 and 16, selected or non-selected measurement values may be displayed using the region 880. According to exemplary embodiments, a non-selected measurement value may be a measurement value whose selection is cancelled by the user.

The ultrasound diagnosis apparatus 1000 may calculate a resultant value based on the selected at least one measurement value and display the calculated resultant value via the display 1400 (S2260). In this case, the resultant value may be displayed in various ways according to exemplary embodiments.

Although an ultrasound diagnosis apparatus and method of measuring an elasticity value using a shear wave have been described throughout the specification, exemplary embodiments described in the specification may be applied to measurement of a time-intensity curve, strain, or hepatic attenuation in ultrasound diagnosis using a contrast agent.

Exemplary embodiments may be implemented through non-transitory computer-readable recording media having recorded thereon computer-executable instructions such as program modules that are executed by a computer. The non-transitory computer-readable recording media may be any available media that can be accessed by a computer and include both volatile and nonvolatile media and both detachable and non-detachable media. Furthermore, the non-transitory computer-readable recording media may include computer storage unit media and communication media. The computer storage unit media include both volatile and nonvolatile and both detachable and non-detachable media implemented by any method or technique for storing information such as computer-readable instructions, data structures, program modules, or other data. The communication media typically embody computer-readable instructions, data structures, program modules, other data of a modulated data signal, or other transmission mechanism, and may include any information transmission media.

The above description is provided for illustration, and it will be understood by those of ordinary skill in the art that various changes in form and details may be made therein without departing from essential features and the spirit and scope of the present inventive concept as defined by the following claims. Accordingly, the above exemplary embodiments and all aspects thereof are examples only and are not limiting.

For example, each component defined as an integrated component may be implemented in a distributed fashion. Likewise, components defined as separate components may be implemented in an integrated manner.

The scope of the present inventive concept is defined not by the detailed description thereof but by the appended claims, and all the changes or modifications within the scope of the appended claims and their equivalents will be construed as being included in the present inventive concept.

What is claimed is:

1. A method of operating an ultrasound diagnosis apparatus, the method comprising:
acquiring a plurality of pieces of elasticity data for an object;
acquiring a plurality of measurement values respectively from the plurality of pieces of elasticity data;
displaying the plurality of measurement values;
selecting at least one of the plurality of measurement values;
displaying the selected at least one measurement value to be distinguished from non-selected measurement values among the plurality of measurement values; and
displaying a resultant value calculated based on the selected at least one measurement value.

2. The method of claim 1, wherein the plurality of measurement values comprise a plurality of elasticity values respectively acquired from the plurality of pieces of elasticity data, and
wherein the displaying of the plurality of measurement values comprises:
determining at least one reference value determined based on the plurality of elasticity values respectively acquired from the plurality of pieces of elasticity data; and
displaying a graph representing the determined at least one reference value and the plurality of measurement values.

3. The method of claim 2, wherein the at least one reference value comprises at least one of a median value, a first quartile, and a third quartile for the plurality of elasticity values.

4. The method of claim 2, wherein the displayed graph comprises at least one baseline representing a magnitude of the at least one reference value.

5. The method of claim 2, wherein the resultant value comprises at least one of a median value, an average value, a reliability index, a standard deviation, and a ratio of interquartile range (IQR)/median value.

6. The method of claim 2, wherein, in the displaying of the graph, indicators respectively representing deviations of the plurality of elasticity values are further displayed.

7. The method of claim 2, wherein the selecting of at least one of the plurality of measurement values comprises:
comparing each of the plurality of measurement values with the determined at least one reference value; and
selecting the at least one measurement value based on a result of the comparing.

8. The method of claim 7, wherein the at least one reference value comprises a first quartile and a third quartile for the plurality of elasticity values, and
wherein the selecting of the at least one measurement value comprises selecting at least one measurement value including an elasticity value that is greater than or equal to the first quartile and less than or equal to the third quartile.

9. The method of claim 2, further comprising calculating reliability indices for the plurality of elasticity values,
wherein the graph further comprises the calculated reliability indices.

10. The method of claim 1, wherein the selecting of the at least one measurement value comprises:
receiving a user input for the plurality of measurement values; and
selecting the at least one measurement value and excluding some measurement values from the plurality of measurement values, based on the user input.

11. An ultrasound diagnosis apparatus comprising:
a controller configured to acquire a plurality of pieces of elasticity data for an object and obtain a plurality of measurement values respectively from the plurality of pieces of elasticity data; and
a display configured to display the plurality of measurement values,
wherein the controller is further configured to select at least one of the plurality of measurement values, control the display to display the selected at least one measurement value to be distinguished from non-selected measurement values among the plurality of measurement values, and control a resultant value calculated based on the selected at least one measurement value to be displayed via the display.

12. The apparatus of claim 11, wherein the plurality of measurement values comprise a plurality of elasticity values respectively acquired from the plurality of pieces of elasticity data,
wherein the controller determines at least one reference value determined based on the plurality of elasticity values respectively acquired from the plurality of pieces of elasticity data, and
wherein the display displays a graph representing the determined at least one reference value and the plurality of measurement values.

13. The apparatus of claim 12, wherein the at least one reference value comprises at least one of a median value, a first quartile, and a third quartile for the plurality of elasticity values.

14. The apparatus of claim 12, wherein the displayed graph comprises at least one baseline representing a magnitude of the at least one reference value.

15. The apparatus of claim 12, wherein the resultant value comprises at least one of a median value, an average value, a reliability index, a standard deviation, and a ratio of interquartile range (IQR)/median value.

16. The apparatus of claim 12, wherein the display further displays indicators respectively representing deviations of the plurality of elasticity values.

17. The apparatus of claim 12, wherein the controller compares each of the plurality of measurement values with the determined at least one reference value and selects the at least one measurement value based on a result of the comparing.

18. The apparatus of claim 17, wherein the at least one reference value comprises a first quartile and a third quartile for the plurality of elasticity values, and
wherein the controller selects at least one measurement value including an elasticity value that is greater than or equal to the first quartile and less than or equal to the third quartile.

19. The apparatus of claim 12, wherein the controller is further configured to calculate reliability indices for the plurality of elasticity values, and
wherein the graph displayed on the display further comprises the calculated reliability indices.

20. The apparatus of claim 11, further comprising an input device configured to receive a user input for the plurality of measurement values,
wherein the controller selects the at least one measurement value and excludes some measurement values from the plurality of measurement values, based on the user input.

21. A non-transitory computer-readable recording medium having recorded thereon a program for performing the method of claim 1.
